(19) United States
(12) Patent Application Publication
(10) Pub. No.: US 2017/0189947 A1
(43) Pub. Date: Jul. 6, 2017

(54) Method and System for Cleaning and Displaying Exercise Mats

(71) Applicant: Fresh Mat, Inc., San Francisco, CA (US)

(72) Inventor: John Butler, San Francisco, CA (US)

(21) Appl. No.: 15/391,420

(22) Filed: Dec. 27, 2016

Related U.S. Application Data
(60) Provisional application No. 62/273,844, filed on Dec. 31, 2015.

(51) Int. Cl. B08B 11/02; A61L 2/00; A63B 21/00; A63B 71/00; A47B 81/00

(52) U.S. Cl. CPC B08B 11/02; A63B 71/0036; A47B 81/00; A63B 21/4037; A61L 2/00

(57) ABSTRACT

Method and apparatus for cleaning storing, moving and displaying exercise mats. A display rack incorporates a rollable base and provides support to one or more rails for hanging an exercise mat. The display rack is designed to slide off of the rollable base into the wash chamber of a cabinet. The wash chamber is used to clean and sanitize the exercise mat hanging on the display rack, after which the display rack can be returned to the rollable base and moved to another location.

### CROSS REFERENCE TO RELATED APPLICATIONS

The present application claims priority to U.S. provisional Patent Application Ser. No. 62/273,844, filed on Dec. 31, 2015, for a “Method and System for Cleaning and Displaying Exercise Mats”, which is herein incorporated by reference in its entirety.

### TECHNICAL FIELD

The disclosed method and system relates to the cleaning and sanitizing of exercise mats and more specifically to a transportable storage rack and associated cleaning cabinet for exercise mats used in a workout center or studio.

### BACKGROUND

Thin, flexible exercise mats are used for a variety of exercise routines, where they provide protection between participants and the floor. Exercise mats are relatively uniform in size, typically 6 feet long and 2 feet wide and can be made of a variety of materials, including, but not limited to rubber, foam or foam wrapped in a cover. Many of the exercise routines where exercise mats are used, such as yoga, Pilates, barre and other routines involve strenuous activity, resulting in sweating, and the accumulation of sweat and other undesirable material on the mats. Additionally, the floors of studios and workout centers are not necessarily clean, so a wet or sticky mat will pick up dirt, bacteria and other waste that is objectionable and potentially unhealthy to exercise participants. Often these mats are provided to participants as a courtesy by a gym, workout center, or exercise studio.

Many patrons will bring their own mats in order to avoid contact with the sweat of others, or they will insist that the studio or workout center clean and sanitize provided mats before use. These businesses are compelled to engage in the labor intensive tasks of displaying, moving, cleaning, sanitizing and drying a large inventory of mats. Gyms, workout centers, or exercise studios have classes scheduled throughout the day with minimal intermission time in order to maximize revenue. In order to provide cleaned and sanitized mats to patrons, it is valuable to have a machine that can perform this function quickly.

Accordingly, there is presently a need for an integrated system to accommodate the combined functions of storing, providing inventory, displaying, moving, batch cleaning, sanitizing and drying exercise mats and associated exercise equipment.

### BRIEF DESCRIPTION OF THE DRAWINGS

The disclosed method and apparatus, in accordance with one or more various embodiments, is described with reference to the following figures. The drawings are provided for purposes of illustration only and merely depict examples of some embodiments of the disclosed method and apparatus. These drawings are provided to facilitate the reader's understanding of the disclosed method and apparatus. They should not be considered to limit the breadth, scope, or applicability of the claimed invention. It should be noted that for clarity and ease of illustration these drawings are not necessarily made to scale.

FIG. 1A illustrates a display rack of the basic cleaning and display system;

FIG. 1B illustrates the components of the basic cleaning and display system;

FIGS. 2A, 2B and 2C provide front views of exercise mats modified for use in the cleaning and display system

FIG. 3A is an expanded view of a hanging rail for use on the cleaning and display system;

FIG. 3B is an alternative hanging rail using indents formed in the rail;

FIG. 3C illustrates an additional alternative hanging rail, wherein the rail is in a serpentine form.

FIG. 4A and FIG. 4B illustrates an adaptation of cleaning and display system to accommodate trays of exercise equipment.

The figures are not intended to be exhaustive or to limit the claimed invention to the precise form disclosed. It should be understood that the disclosed method and apparatus can be practiced with modification and alteration, and that the invention should be limited only by the claims and the equivalents thereof.

### DETAILED DESCRIPTION

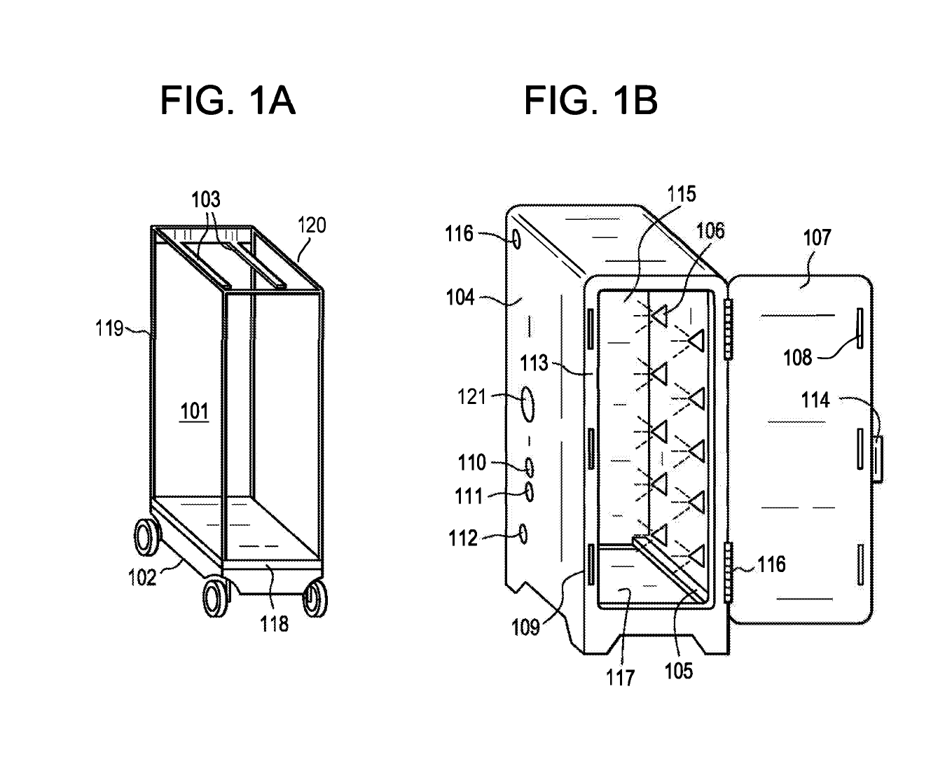

Referring now to the drawings, and particularly to FIGS. 1A and 1B thereof, associated components of a mat cleaning and display system are illustrated. Referring to FIG. 1B, a display rack 101 comprises a framework to support exercise mats. In some cases, the rack includes a bottom member 118 designed to provide stability and to releasably attach to a rollable base 102 incorporating wheels. The display rack 101 includes one or more vertical members 119 to support at least one horizontal member 120 at the top of the display rack 101. The vertical member 119 is designed to provide one or more hanging rails 103 for exercise mats. Alternatively, rollable base 102 could include the vertical members 119 supporting the one or more horizontal members 120, so that the horizontal members 120 would then be releasably attached to the vertical members 119 supporting hanging points 103 of display rack 101.

A bottom member 118 of FIG. 1A is configured to stay relatively secure on a movable base 102, while allowing the bottom member 118 to be slid from the base 102 into a wash chamber 117. Alternative arrangements for the bottom member 118 include, but are not limited to, a plastic or Teflon cover, wheels, or bearings to allow for easy movement of display rack 101 off of the base 102. In one such case, the bottom member 118 can be secured with a moving or static locking arrangement to hold the display rack 101 in place on the moveable base 102 when the display rack 101 is being used for display or is being moved. Base 102 is configured with wheels to allow for easy movement of the empty or loaded display rack 101 when attached to the base 102.

Referring again to FIG. 1B, in one case, an outer cabinet 104 has three interconnected vertical side walls and interconnected top and bottom walls. The interconnected top and bottom walls form a vertical access opening for the admission of the display rack 101 into, and removal of display rack 101 from, a wash chamber 115. This access opening is provided with a door 107 pivotally secured to one wall of the cabinet 104 by a hinge member 116. The door 107 includes a handle 114 and latching member 108 for providing a watertight and secure connection to cabinet 104 when in a closed position, using additional latching member 109. Alternatively, the door 107 could be a roll-down or roll-up segmented door using a guide and seal 113 on the periphery of the vertical access opening of the cabinet 104. The lower extremity of the wash chamber 115 is defined by the bottom wall 117, the periphery of which is in liquid-tight relationship to the three vertical walls of the cabinet 104 and the door 107 when the door 107 is closed. The bottom wall 117 has a centrally depressed portion forming a sump in which liquid will collect. A support member 105 is adapted to support the display rack 101 when the display rack 101 is released from moveable base 102 and placed into wash chamber 115. The support member 105 allows the display rack 101 to slide into wash chamber 117. In some cases, the support member 105 is adapted with a Teflon or plastic cover, wheels, rollers or bearings or any other suitable method to allow the display rack 101 and the bottom member 118 of FIG. 1A to freely slide into wash chamber 117.

Again referring to FIG. 1B, wash chamber 115 includes one or more fixed spray nozzles 106 on at least one of the interior walls of the wash chamber 115. The fixed spray nozzle 106 could be augmented by, or replaced with, a rotating reaction-type spray arm, a motor driven spray arm or by one or more pressure driven or motor driven brushes attached to the interior walls of the wash chamber 115. Wash chamber 115 may also include ultra-violet (U.V.) light or ozone sources on one or more of its walls to assist in sanitizing the exercise mats.

The cabinet 104 of FIG. 1B includes an inlet 110, an outlet 111 and an electrical input 112 through one or more of its walls. The inlet 110 is adapted for use with water and/or various cleaning and/or sanitizing agents, including, but not limited to water of varying pH, ionized water, low alkalinity water, high alkalinity water, water with stabilized probiotics and water treated with an electrochemical process to produce hypochlorous or hypobromous acid. The wash chamber 115 could additionally be adapted with an internal or external mechanism to provide for filtering and/or reuse of cleaning and sanitizing materials. The inlet 121 is adapted to supply high pressure gas, such as air, ionized gas or inert gas such as nitrogen to the spray nozzle 106, where it could be combined with the contents of the inlet 110 to augment the cleaning process. An optional input and output 116 and 119 could also be included to provide air ingress and egress, respectively, to the cabinet 104 for drying wet mats. It should be understood that the drying mechanism of the cabinet 104 could include a variety of structures that provide drying using known methods, including any of the various embodiments of the combined condensation and heat pump of US20140238450 to Bertram, which is hereby incorporated herein by reference. The cabinet 104 could additionally be adapted to include a motor pump assembly within the bottom wall, or the motor and pump might alternatively be provided external to the cabinet 104. Additionally, the cabinet 104 could include mechanical and/or electronic control elements for the mat washing cycles along with one or more displays to monitor system operation.

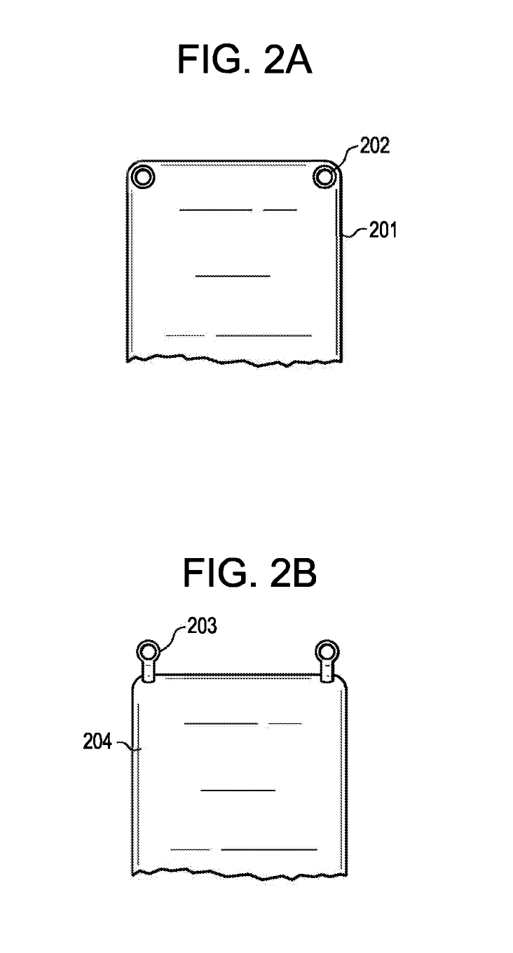

FIG. 2A illustrates an exercise mat adapted for use with an embodiment of the display rack 101. In FIG. 2A grommet 202 is added to the exercise mat 201 such that the exercise mat 201 can be hung from a hanging rail 103 of FIG. 1. FIG. 2B illustrates an exercise mat 205 with a clip 203 for use with the hanging rail 103 of FIG. 1.

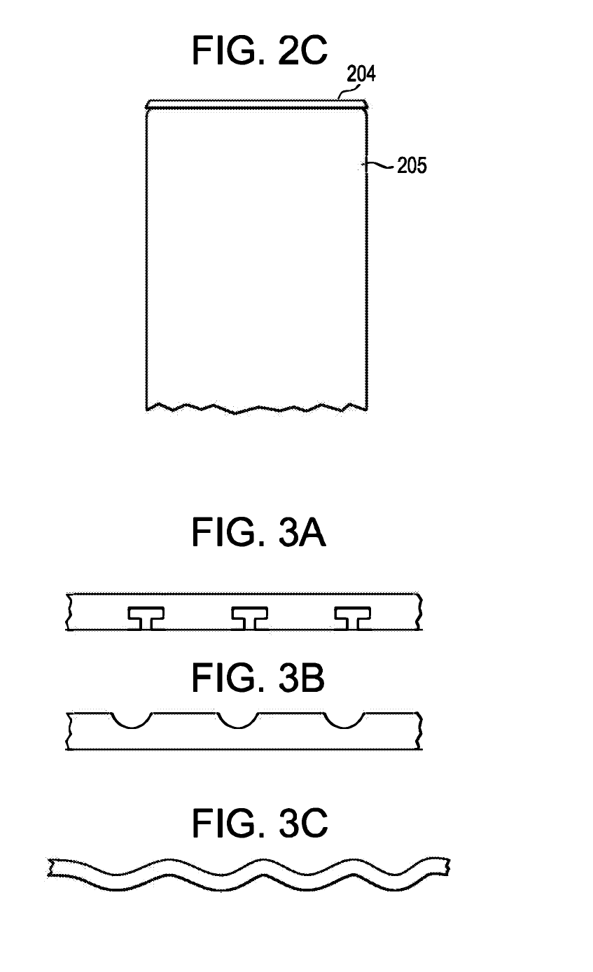

FIG. 2C illustrates an exercise mat adapted with a T-slot key 204, for hanging the mat 204 from the hanging rail 103 illustrated in FIG. 1, wherein the hanging rail 103 has been modified with the T-slot hanger 302 illustrated in FIG. 3A. When using the arrangement of FIGS. 2C and 3a the mats would be loaded from the side of rack 101 of FIG. 1 instead of the front loaded arrangement shown in FIGS. 1, 3B and 3C.

The hanging rail 103 could be a straight element as shown in FIG. 1. Alternatively, in order to keep the mats at a fixed distance from each other during the cleaning process, the hanging rail 103 of FIG. 1 could be provided with indents illustrated in FIG. 3B or could be formed in a serpentine manner as shown in FIG. 3C.

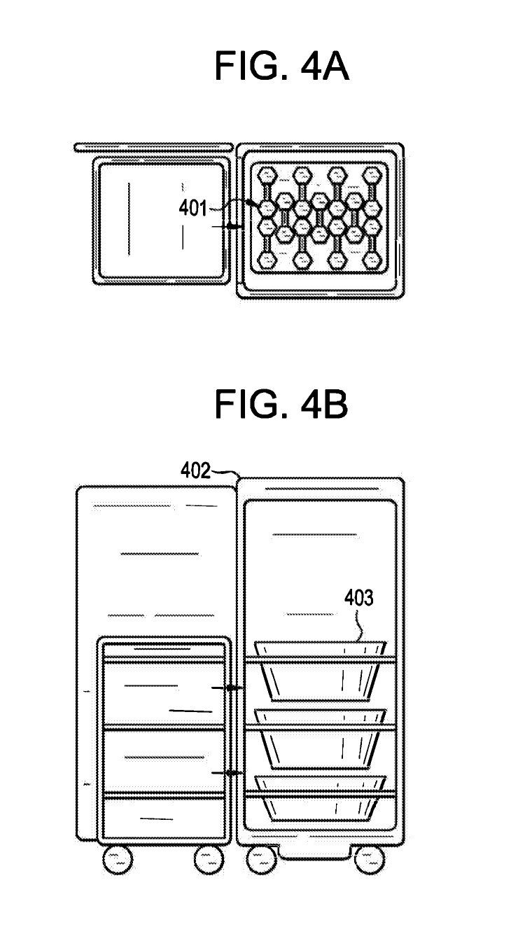

FIG. 4A illustrates a tray 401 adapted for use in a wash chamber 402. The wash chamber 402 has been adapted for admission of tray 401 into and out of the wash chamber. The wash chamber 402 may be further adapted to accommodate a plurality of trays simultaneously. As shown in FIG. 4B, the wash chamber 402 supports a tray 403 using parallel rails attached to the vertical side walls of the wash chamber 402. As would be understood by one skilled in the art, the tray 401 could also be supported using mechanisms in addition to the parallel rails shown, including trays 401 adapted to being supported by the sides of the wash chamber 402. The tray 401 provides containment of exercise equipment, such as small dumb-bells and exercise bands that may become sweaty and otherwise contaminated while in use. Use of the tray 401 allows the mat cleaning system to be used for equipment cleaning when not being used to clean exercise mats.

By way of example only, when in use for storage or display of exercise mats the display rack would be secured on the rollable base. Patrons could then remove the mats for use in the exercise environment, during which the display rack could be moved for easy access to patrons or staff to hang the soiled exercise mats on the display rack. Once the soiled exercise mats are hung on the display rack staff can roll the display rack to the cleaning cabinet, where staff could release the display rack from the rollable base and push it into the wash chamber of the cleaning cabinet. The door would then be closed and the wash sequence could commence. Upon completion of the cleaning and optional drying sequence staff could open the door of the cleaning cabinet, replace and secure the display rack on the rollable base and roll the display rack with the clean exercise mats back to a display or storage areas for immediate use.

Although the disclosed method and apparatus is described above in terms of various examples of embodiments and implementations, it should be understood that the particular features, aspects and functionality described in one or more of the individual embodiments are not limited in their applicability to the particular embodiment with which they are described. Thus, the breadth and scope of the claimed invention should not be limited by any of the examples provided in describing the above disclosed embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide examples of instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

A group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although items, elements or components of the disclosed method and apparatus may be described or claimed in the singular, the plural is contemplated to be within the scope thereof unless limitation to the singular is explicitly stated.

The presence of broadening words and phrases such as “one or more,” “at least,” “but not limited to” or other like phrases in some instances shall not be read to mean that the narrower case is intended or required in instances where such broadening phrases may be absent.

Additionally, the various embodiments set forth herein are described with the aid of block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. A system for cleaning and displaying exercise mats, comprising:
a display rack with at least one vertical support member, a bottom member and a horizontal member, the bottom member being releasably secured to a rollable base and the horizontal support member is supports to one or more rails for hanging an exercise mat;
a cabinet having three vertical walls and top and bottom walls defining a wash chamber with an access opening, the cabinet having a door to cover the access opening and a support member on the bottom wall of the wash chamber for supporting admission of the display rack into the wash chamber when the display rack is released from the rollable base;
a liquid delivery device located within the wash chamber such that liquid circulated from the liquid delivery device is distributed throughout the wash chamber;
wherein the rollable base is sized at its uppermost surface to approximately match the height of the support member on the bottom wall of the wash chamber, such that the display rack can be admitted into the wash chamber when released from the rollable base.

2. The system of claim 1, wherein the rail is a straight bar sized to fit within an opening on the exercise mat such that the exercise mat hangs from the rail when inserted thereon.

3. The system of claim 2, wherein the rail is notched at regular intervals.

4. The system of claim 1, wherein the rail is a serpentine bar sized to accept an opening on the exercise mat such that the exercise mat hangs from the rail when inserted thereon.

5. The system of claim 1, wherein the rail and exercise mat have a slot and key relationship relative to each other, such that an exercise mat with a corresponding key or slot will hang therefrom.

6. The system of claim 1, wherein the liquid delivery device is a fixed spray nozzle.

7. The system of claim 1, wherein the liquid delivery device is a rotating spray arm.

8. The system of claim 7, wherein the liquid delivery device is a reaction-type spray arm.

9. The system of claim 1, wherein a rotating brush is located within the wash chamber, such that the rotating brush can be used to physically cleanse the surface of an exercise mat in the wash chamber.

10. The system of claim 9, wherein the rotating brush is pressure driven.

11. The system of claim 1, wherein a non-liquid sanitization element selected from the group consisting of U.V. illumination, ozonolysis, or disinfecting aerosol is used in addition to liquid cleansing.

12. The system of claim 1, wherein the rollable base is adapted to be releasably secured to the bottom member of the display rack using a latch.

13. The system of claim 1, wherein the rollable base is adapted to be releasably secured to the bottom member of the display rack without the use of a latch.

14. The system of claim 1, wherein the surface of the rollable base is adapted to facilitate sliding of the display rack thereon.

15. The system of claim 1, wherein the surface of the rollable base has rolling elements to facilitate sliding of the display rack thereon.

16. The system of claim 1, wherein the display rack has rolling elements on the bottom member to facilitate sliding of the display rack into the wash chamber.

17. The system of claim 1, wherein the wash chamber has one or more support members attached longitudinally to one or more vertical walls, such that the support members can support trays admitted into the wash cabinet.

18. The system of claim 1, wherein the cabinet has one or more ports for ingress and egress of pressurized gas.

19. The system of claim 1, wherein the liquid delivery device is assisted by pressurized gas.

20. A display rack for exercise mats, comprising:
at least one vertical support member;
a bottom member;
a horizontal member supporting one or more rails for hanging an exercise mat;
a rollable base;
wherein the bottom member is releasably secured to the rollable base
and wherein the top of the rollable base is sized to approximately match the height of a support member on the bottom wall of a wash chamber, such that the display rack can be admitted into the wash chamber when released from the rollable base.

21. A system for cleaning storing, moving and displaying exercise mats, comprising:
a display rack with at least one vertical support member, a bottom member and a horizontal member, the bottom member being releasably secured to a rollable base and the horizontal support member is supports to one or more rails for hanging an exercise mat;
a cabinet having three vertical walls and top and bottom walls defining a wash chamber with an access opening, the cabinet having a door to cover the access opening and a support member on the bottom wall of the wash chamber for supporting admission of the display rack into the wash chamber when the display rack is released from the rollable base;
a liquid delivery device located within the wash chamber such that liquid circulated from the liquid delivery device is distributed throughout the wash chamber;
wherein the rollable base is sized at its uppermost surface to approximately match the height of the support member on the bottom wall of the wash chamber, such that the display rack can be admitted freely into the wash chamber when released from the rollable base.

22. The system of claim 21, wherein the cabinet includes a motor pump assembly for circulating liquid through the liquid delivery device and to facilitate the egress of liquid out of the wash chamber.
